(19) United States
(12) Patent Application Publication
(10) Pub. No.: US 2001/0031875 A1
(43) Pub. Date: Oct. 18, 2001

(54) Optically active ionic liquid

(76) Inventor: Tomoya Kitazume, Tokyo (JP)

Correspondence Address: CROWELL & MORING LLP, Intellectual Property Group, P. O. Box 14300, Washington, DC, 20044-4300, US

(21) Appl. No.: 09/799,098

(22) Filed: Mar. 6, 2001

(30) Foreign Application Priority Data
Mar. 6, 2000 (JP) 2000-061043

(51) Int. Cl. C07D 213/20

(52) U.S. Cl. 546/347

(57) ABSTRACT

The invention relates to an optically active ionic liquid containing (a) a cationic species having a heterocyclic ring; and (b) an anionic species. This cationic species contains at least one asymmetric carbon atom. This ionic liquid can be produced by a process including the step of reacting a heterocyclic compound, which contains an asymmetric carbon atom, with an alkylation agent. The ionic liquid can be useful as a solvent for asymmetric syntheses.

### BACKGROUND OF THE INVENTION

The present invention relates to ionic liquids, which can serve as both of catalyst and solvent. Thus, much attention has recently been attracted in organic syntheses to ionic liquids. Ionic liquids may have affinity for particular substances and may have a function as catalyst. Thus, ionic liquids may be used as reaction solvents and furthermore may also be used for the purpose of increasing the process efficiency, for example, in separation of substances.

Inorganic Chemistry, Vol. 35, No., 1996 discloses an ionic liquid, 1-ethyl-3-methylimidazolium=trifluoromethanesulfonate, and its analogous substances.

### SUMMARY OF THE INVENTION

It is an object of the present invention to provide a novel ionic liquid.

According to the present invention, there is provided an optically active ionic liquid. This ionic liquid may comprise (a) a cationic species comprising a heterocyclic ring; and (b) an anionic species. Furthermore, the ionic liquid may comprise an asymmetric carbon atom.

### DESCRIPTION OF THE PREFERRED EMBODIMENTS

An optically active ionic liquid according to the invention can be useful as a solvent for asymmetric syntheses. The ionic liquid can be defined as having a property of ionic liquid and as having an optical activity derived from an asymmetric carbon atom.

The ionic liquid can be a salt formed of a cationic species and an anionic species. Furthermore, it can be defined as having a melting point of about 100° C. or lower and as having a vapor pressure that is substantially zero at about room temperature (e.g., 25° C.). Thus, it can be defined as being a substance that is in the form of liquid until a high temperature that may be about 200° C., preferably 300° C. The ionic liquid can be used as a solvent for organic and inorganic compounds, as a catalyst for alkylation, polymerization, oligomerization and the like, and as an extraction solvent for metals and organic matters.

The ionic liquid can have an optical activity derived from asymmetric carbon atom. Thus, it is possible to synthesize an optically active compound from an optically inactive raw material using the ionic liquid through selective solubility and selective catalytic unction of the ionic liquid. The ionic liquid can be used in chromatography due to its selective solubility to achieve optical resolution and due to its selective adsorption. It may be possible to use the ionic liquid in further various uses.

As stated above, the ionic liquid can be defined as containing (a) a cationic species having a heterocyclic ring; and (b) an anionic species. This anionic species can be selected from a carboxylate anion represented by the general formula (1), an alkanesulfonate anion represented by the general formula (2), and a bis(alkanesulfonyl)amide anion represented by the general formula (3),

RCOO⁻ (1)

where R is an alkyl group or fluoroalkyl group,

RSO₃⁻ (2)

where R is defined as above,

(RSO₂)₂N⁻ (3)

where R is defined as above, or a combination of two of R is an alkylene group or fluoroalkylene group.

In the carboxylate anion represented by the general formula (1), the carbon atom number of R may be of about 1-10. Its examples include acetate anion, propionate anion, trifluoroacetate anion, pentafluoropropanoate anion, heptafluorobutanoate anion, and nonafluoropentanoate anion.

In the alkanesulfonate anion represented by the general formula (2), the carbon atom number of R may be of about 1-10. Its examples include methanesulfonate anion, ethanesulfonate anion, trifluoromethanesulfonate anion, pentafluoroethanesulfonate anion, 2,2,2-trifluoroethanesulfonate anion, heptafluoropropanesulfonate anion, 2,2,3,3,3-pentafluoropropanesulfonate anion, and nonafluorobutanesulfonate anion.

In the bis(alkanesulfonyl)amide represented by the general formula (3), the carbon atom number of R may be of about 1-10. Its examples include bis(trifluoromethanesulfonyl)amide anion, bis(pentafluoroethanesulfonyl)amide anion, bis(heptafluoropropanesulfonyl)amide anion, bis(nonafluorobutanesulfonyl)amide anion, bis(undecafluoropentanesulfonyl)amide anion, (trifluoromethanesulfonyl)(pentafluoroethanesulfonyl)amide anion, (trifluoromethanesulfonyl)(heptafluoropropanesulfonyl)amide anion, (trifluoromethanesulfonyl)(nonafluorobutanesulfonyl) amide anion, bis(2,2,2-trifluoroethanesulfonyl)amide anion, (trifluoromethanesulfonyl)(2,2,2-trifluoroethanesulfonyl) amide anion, and an anion represented by the following formula:

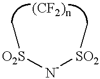

where n is an integer of 2-10.

Further examples of the anionic species include halogen ions and complex ions, for example, hexafluorophosphate, tetrachloroaluminate, and hexafluoroborate.

Of the above-mentioned exemplary anionic species, fluorocarboxylate anion, fluoroalkanesulfonate anion and bis(fluoroalkanesulfonyl)amide anion are preferable. Furthermore, perfluoroalkanesulfonate anion and bis(perfluoroalkanesulfonyl)amide anion are more preferable.

The cationic species of the ionic liquid is defined as being a positively charged chemical species of a heterocyclic compound containing at least one asymmetric carbon atom. This heterocyclic compound may have a base heterocyclic ring selected from pyrrole ring, 2-pyrroline ring, pyrrolidine ring, pyrazole ring, 2-pyrazoline ring, pyrazolidine ring, imidazole ring, 2-imidazoline ring, imidazolidine ring, 1H-1,2,3-triazole ring, 2H-1,2,3-triazole ring, 1H-1,2,4-triazole ring, 4H-1,2,4-triazole ring, 1H-tetrazole ring, isoxazole ring, oxazole ring, furazane ring, isothiazole ring, thiazole ring, pyridine ring, piperidine ring, pyridazine ring, pyridimine ring, pyradine ring, piperazine ring, 1,3,5-triazine ring, 1,2,4,5-tetrazine ring, 1,4-oxazine ring, morpholine ring, 1,4-thiazine ring, indole ring, indoline ring, isoindole ring, isoindoline ring, 1H-indazole ring, 2H-indazole ring, benzoimidazole ring, benzotriazole ring, benzoxazole ring, benzothiazole ring, benzothiazoline ring, purine ring, quinoline ring, isoquinoline ring, cinnoline ring, quinazoline ring, quinoxaline ring, phthaladine ring, 1,8-naphthyridine ring, pteridine ring, carbazole ring, acridine ring, phenazine ring, phenoxadine ring, phenothiadine ring, phenantridine ring, 1,10-phenanthroline ring, phenazone ring, and quinuclidine ring.

When the ionic liquid is formed, it becomes stable by an ionic bond between the cationic species and the corresponding anionic species. In fact, this ionic bond can be formed, when a nitrogen atom(s) contained in the base heterocyclic ring is positively charged, or when the electric charge is delocalized over the whole heterocyclic ring.

A group(s) of an arbitrary structure can be bonded to the base heterocyclic ring. It is preferable that the ionic liquid contains at least one asymmetric carbon atom in the cationic species. The at least one asymmetric carbon may be contained in the base heterocyclic ring having a cationic structure or may be contained in a structural portion (which is other than the base heterocyclic ring) of the cationic species.

A nitrogen-containing structure of the cationic species can be one represented by the general formula (4) or (5),

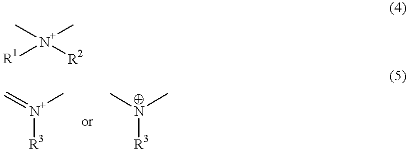

where R¹, R² and R³ are the same or different alkyl groups, fluoroalkyl groups or aryl groups. When the base heterocyclic ring contains at least two nitrogen atoms, each nitrogen atom can have a structure represented by the general formula (4) or (5).

The alkyl group for R¹, R² and R³ is preferably one having a carbon atom number of about 1-22, more preferably about 1-6, and optionally having a substituent. Examples of such alkyl group are methyl group, ethyl group, n-propyl group, isopropyl group, n-butyl group, isobutyl group, tert-butyl group, pentyl group, isopentyl group, dodecyl group, tetradecyl group, octadecyl group, and benzyl group. The fluoroalkyl group for R¹, R² and R³ is preferably one having a carbon atom number of about 1-22, more preferably about 1-6, and optionally being branched. Examples of such fluoroalkyl group are trifluoromethyl group, pentafluoroethyl group, 2,2,2-trifluoroethyl group, heptafluoropropyl group, 1,1,1,3,3-hexafluoropropyl group, 2,2,3,3,3-pentafluoropropyl group, and nonafluorobutyl group Examples of the aryl group for R¹, R² and R³ are phenyl group and substituted phenyl groups having substituents (e.g., the above alkyl groups).

An ionic liquid having a cationic species represented by the general formula (4), in which R¹ and R² are different groups, has a tendency to have a melting point that is lower than that of another ionic liquid having a cationic species in which R¹ and R² are the same groups. Suitable groups can be chosen for a combination of R¹ and R², depending on the type of the cationic species, particularly the base heterocyclic ring structure. Preferable combinations of R¹ and R² in the general formula (4) are methyl group (R¹) and ethyl group (R²), methyl group (R¹) and n-propyl group (R²), methyl group (R¹) and isopropyl group (R²), and methyl group (R¹) and n-butyl group (R²).

In case that the bass heterocyclic ring contains at least two nitrogen atoms and that the electric charge is delocalized, it is preferable that R³ bonded to the nitrogen atoms are different groups. With this, it is possible to obtain an ionic liquid having a lower melting point.

An example of the ionic liquid can be a pyrrolidine derivative represented by the general formula (6),

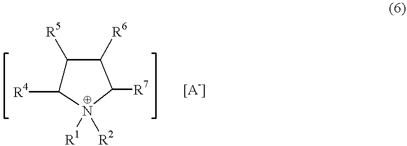

where R¹ and R² are defined as above; R⁴, R⁵, R⁶ and R⁷ are monovalent groups; and A⁻ is an anion. These R¹ and R² can be selected from the above-mentioned preferable examples. In the general formula (6), carbon atom to which R⁴, R⁵, R⁶ or R⁷ is bonded can be an asymmetric carbon atom. Furthermore, R⁴, R⁵, R⁶ and R⁷ can independently be alkyl groups, fluoroalkyl groups, aryl group, heterocyclic group, and hydrogen atoms. All of these R⁴, R⁵, R⁶ and R⁷ are, however, not hydrogen atoms at the same time. The alkyl group for R⁴, R⁵, R⁶ and R⁷ is preferably one having a carbon atom number of about 1-22, more preferably about 1-6, and optionally having a substituent. Examples of such alkyl group can be the same as those of the alkyl group for R¹, R² and R³. The fluoroalkyl group for R⁴, R⁵, R⁶ and R⁷ is preferably one having a carbon atom number of about 1-22, more preferably about 1-6, and optionally being branched. Examples of such fluoroalkyl group can be the same as those of the fluoroalkyl group for R¹, R² and R³. Examples (hydrocarbon groups) of the aryl group for R⁴, R⁵, R⁶ and R⁷ are phenyl group and substituted phenyl groups having substituents (e.g., the above alkyl groups). Exemplary heterocyclic groups for R⁴, R⁵, R⁶ and R⁷ are pyridyl group, piperidyl group, piperazinyl group, pyrimidyl group, imidazolyl group, pyrrolyl group, pyrrolinyl group, pyrrolidyl group, and substituted groups containing, for example, the above alkyl groups substituted for hydrogen atoms of these exemplary hetero aryl groups.

Of the above-mentioned examples of the ionic liquid, a preferable example can be a nicotine analogue derivative represented by the general formula (7)

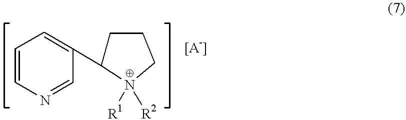

where R¹ and R² are defined as in the general formula (4); and A⁻ is an anion. In the general formula (7), R¹ and R² are preferably alkyl groups or fluoroalkyl groups. Carbon atom to which the pyridyl group is bonded is an asymmetric carbon atom. The alkyl group for R¹ and R² is preferably one having a carbon atom number of about 1-22, more preferably about 1-6, and optionally having a substituent. Examples of such alkyl group can be the same as those of the alkyl group for R¹, R² and R³ of the general formulas (4) and (5). The fluoroalkyl group for R¹ and R² is preferably one having a carbon atom number of about 1-22, more preferably about 1-6, and optionally being branched. Examples of such fluoroalkyl group can be the same as those of the fluoroalkyl group for R¹, R² and R³ of the general formulas (4) and (5).

In the general formula (7), it is preferable that R¹ and R² are different groups. Suitable groups can be chosen for a combination of R¹ and R², depending on the type of the cationic species, particularly the base heterocyclic ring structure. Preferable combinations of R¹ and R² of the general formula (7) can be the same as those of R¹ and R² of the general formula (4).

Exemplary anionic species include the above-mentioned ones represented by the general formulas (1), (2) and (3), hexafluorophosphate anion, tetrachloroaluminate anion, and hexafluoroborate anion. Of these, fluorocarboxylate anion, fluoroalkanesulfonate anion, and bis(fluoroalkanesulfonyl)amide anion are preferable. Furthermore, perfluoroalkanesulfonate and bis(perfluoroalkanesulfonyl)amide anion are more preferable. In particular, trifluoromethanesulfonate anion and bis(trifluoromethanesulfonyl)amide anion are preferable.

The process for producing the ionic liquid is not particularly limited. It is exemplarily described as follows. At first, a quaternary ammonium salt can be obtained by reacting a heterocyclic compound (a secondary or tertiary amine) having the above-mentioned asymmetric carbon, with an alkylation agent. Typical examples of the alkylation agent are methyl chloride, ethyl chloride, methyl bromide, ethyl bromide, dimethyl sulfate, diethyl sulfate, and benzyl chloride.

Furthermore, the alkylation agent can be selected from other conventional alkylation agents containing moieties corresponding to R¹, R² or R³. The quaternary ammonium salt obtained by the alkylation contains anionic species such as a halogen ion (e.g., chlorine ion) or sulfate ion. Therefore, it is possible to obtain, for example, a trifluoromethanesulfonate by adding trifluoromethanesulfonic acid to the resulting quaternary ammonium salt to achieve ion-exchange of the anionic species. For example, an anionic species exchange from a halogen ion to bis(perfluoroalkanesulfonyl)amide ion can be achieved by reacting the quaternary ammonium salt with bis(trifluoromethanesulfonyl)amide lithium in water. Another anionic species exchange from a halogen ion to perfluoroalkanesulfonate ion can be achieved by reacting the quaternary ammonium salt with a potassium salt of perfluoroalkanesulfonic acid. A still another anionic species exchange from a halogen ion to a carboxylic acid ion or perfluorocarboxylic acid ion can be achieved by reacting the quaternary ammonium salt with a salt of a metal (e.g., sodium, potassium or silver). In this case, the ionic liquid can be obtained by removing an insoluble metal halide as a by-product. Furthermore, the ionic liquid can be obtained by only an alkylation using a particular alkylation agent, that is, an alkyl ester (e.g., ethyl trifluoromethanesulfonate) of an acid (e.g., trifluoromethanesulfonic acid) corresponding to an anionic species of the ionic liquid. With this, the resulting ionic liquid can contain an anionic species of trifluorosulfonate or the like.

The alkylation may be conducted at a temperature of 20-150° C., preferably about 60-100° C. The alkylation can be conducted under a pressure of about 1.0 MPa or lower in case that a low-boiling-point alkylation agent (e.g. methyl chloride) is used in the reaction. It is, however, usually possible to conduct the alkylation under atmospheric pressure. It is preferable to conduct the alkylation in a solvent such as a chlorine-containing solvent (e.g., chloroform, methyl chloride and trichloroethane) or an alcohol solvent (e.g., ethanol and isopropanol).

The following nonlimitative examples are illustrative of the present invention.

### EXAMPLE

### Production of (−)-N-ethylnicotiniumbis(trifluoromethanesulfonyl)amide

A chloroform solution (10 ml) containing 1.62 g (10 mmol) of S-nicotine and 1.09 g (10 mmol) of ethyl bromide was refluxed for 4 hours, followed by distilling chloroform out under reduced pressure. The resulting product was washed with diethyl ether three times. Then, water (30 ml) was added to the residue. After that, 2.87 g (10 mmol) of bis(trifluoromethanesulfonyl)imide lithium (Tf₂NLi) were added, followed by stirring for 6 hours at 70° C. and then lowering the temperature to room temperature. The product was extracted with methylene chloride, followed by distilling the solvent out under reduced pressure, thereby obtaining the target product. The analytical data of the target product were as follows.

Angle of rotation: [α]_{D}²⁰-66 (c, 1.247, CHCl₃).

¹HNMR (CDCl₃): δ 1.70 (3H, t, J=7.42 Hz), 1.89-1.94 (2H, m) 2.25 (3H, s), 2.35-2.51 (2H, m), 3.24-3.30 (1H, m), 3.52 (1H, t, J=7.97 Hz), 4.68 (2H, q, J=7.42 Hz) 7.99 (1H, dd, J=6.04, 7.97 Hz) 8.46 (1H, d, J=7.96 Hz) 8.71 (1H, s), 8.74 (1H, d, J=6.04 Hz).

¹⁹FNMR (CDCl₃): δ 82.74 (standard: C₆F₆).

¹³CNMR (CDCl₃): δ 16.532, 23.137, 35.607, 40.243, 56.632, 57.626, 66.818, 119.577 (CF₃, q, J=320.6 Hz), 128.298, 142.149, 142.392, 144.061, 147.172.

IR (γ): 3076, 2952, 2793, 1635 cm⁻¹.

### Reaction Example

152 mg (1 mmol) of 4-methoxy(1′-methyl)benzyl alcohol, 0.111 ml (1.2 mmol) of vinyl acetate, and 40 mg of lipase PS (Pseudomonas cepacia of Amano Seiyaku Co.) were added to 2 ml of the optically active ionic liquid obtained in Example, followed by stirring for 28 hours at room temperature. After that, 36 mg of the acetate form and 99 mg of 4-methoxy(1′-methyl)benzyl alcohol were obtained using a column chromatograph. The total recovery of these was 83%.

Using a gas chromatograph, the acetate form and 4-methoxy(1′-methyl)benzyl alcohol were found to respectively have optical purities of 69% ee and 35% ee.

The entire disclosure of Japanese Patent Application No. 2000-061043 filed on Mar. 6, 2000, including specification, claims and summary, is incorporated herein by reference in its entirety.

What is claimed is:

1. An optically active ionic liquid.

2. An optically active ionic liquid comprising:
a cationic species comprising a heterocyclic ring; and
an anionic species.

3. An optically active ionic liquid comprising an asymmetric carbon atom.

4. An ionic liquid according to claim 2, wherein said anionic species is selected from the group consisting of a carboxylate anion represented by the general formula (1), an alkanesulfonate anion represented by the general formula (2), and a bis(alkanesulfonyl)amide anion represented by the general formula (3),
RCOO⁻ (1)
where R is an alkyl group or fluoroalkyl group,
RSO₃⁻ (2)
where R is defined as above,
(RSO₂)₂N⁻ (3)
where R is defined as above, or a combination of two of R is an alkylene group or fluoroalkylene group.

5. An ionic liquid according to claim 2, wherein said anionic species comprises a fluorocarboxylate anion, a fluoroalkanesulfonate anion, or a bis(fluoroalkanesulfonyl) amide anion.

6. An ionic liquid according to claim 2, wherein said anionic species comprises a perfluoroalkanesulfonate anion or a bis(perfluoroalkanesulfonyl)amide anion.

7. An ionic liquid according to claim 2, wherein said cationic species is a cationic species of an optically active heterocyclic compound comprising at least one asymmetric carbon atom.

8. An ionic liquid according to claim 2, wherein said heterocyclic ring comprises a structure represented by the general formula (4) or (5) 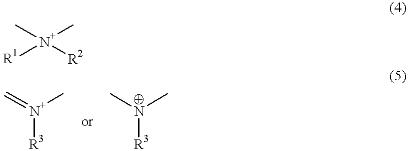
where R¹, R² and R³ are the same or different alkyl groups, fluoroalkyl groups or aryl groups.

9. An ionic liquid according to claim 8, wherein said alkyl groups have a carbon atom number of about 1-22 in the molecule and optionally comprise a substituent,
wherein said fluoroalkyl groups have a carbon atom number of about 1-22 in the molecule and optionally comprise a branch, and
wherein said aryl groups comprise a phenyl group and substituted phenyl groups.

10. An ionic liquid according to claim 8, wherein said R¹ and R² are different alkyl groups, fluoroalkyl groups or aryl groups.

11. An ionic liquid according to claim 10, wherein said R¹ is methyl group, and said R² is one selected from the group consisting of ethyl group, n-propyl group, isopropyl group and n-butyl group.

12. An ionic liquid according to claim 2, which is a pyrrolidine derivative represented by the general formula (6), 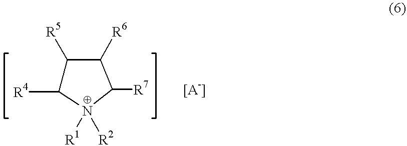
where R¹ and R² are the same or different alkyl groups, fluoroalkyl groups or aryl groups; R⁴, R⁵, R⁶ and R⁷ are monovalent groups, and A⁻ is an anion.

13. An ionic liquid according to claim 12, wherein R⁴, R⁵, R⁶ and R⁷ are independently alkyl groups, fluoroalkyl groups, aryl groups, heterocyclic groups or hydrogen atoms, and wherein all of R⁴, R⁵, R⁶, and R⁷ are not hydrogen atoms at the same time.

14. An ionic liquid according to claim 2, which is a compound represented by the general formula (7), 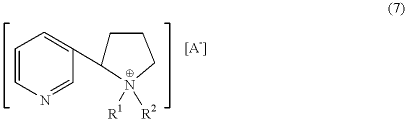
where R¹ and R² are the same or different alkyl groups, fluoroalkyl groups or aryl groups; and A⁻ is an anion.

15. An ionic liquid according to claim 2, which is an optically active N-ethylnicotiniumbis(trifluoromethanesulfonyl)amide or mixtures thereof.

16. A process for producing an optically active ionic liquid, said optically active ionic liquid comprising:
a cationic species comprising a heterocyclic ring; and
an anionic species,
said process comprising reacting a heterocyclic compound comprising an asymmetric carbon atom, with an alkylation agent.

17. A process according to claim 16, further comprising subjecting a product of said reacting to an anion exchange.

18. A process according to claim 16, wherein said heterocyclic compound is a secondary or tertiary amine, and a product of said reacting is a quaternary ammonium salt.

19. A process according to claim 17, wherein said alkylation agent comprises a halogen ion as an anionic species,
wherein said anionic species of said ionic liquid is a bis(perfluoroalkanesulfonyl)amide ion, and
wherein said anion exchange from said halogen ion to said bis(perfluoroalkanesulfonyl)amide ion is conducted by reacting said product with bis(trifluoromethanesulfonyl)amide lithium in water.

20. A process according to claim 16, wherein said alkylation agent comprises an anionic species that is identical with said anionic species of said ionic liquid.
